(19) United States
(12) Patent Application Publication
(10) Pub. No.: US 2007/0104608 A1
(43) Pub. Date: May 10, 2007

(54) Method for the cooling of cleaned items in automatic cleaning and disinfecting machines

(75) Inventors: Bruno Gaus, Offenburg (DE); Denis Lehmann, Ortenberg (DE); Thomas Nager, Offenburg (DE)

Correspondence Address: BIRCH STEWART KOLASCH & BIRCH, PO BOX 747, FALLS CHURCH, VA, 22040-0747, US

(73) Assignee: MEIKO Maschinenbau GmbH & Co KG, Offenburg (DE)

(21) Appl. No.: 10/576,204

(22) PCT Filed: Oct. 18, 2004
(86) PCT No.: PCT/EP04/11709

§ 371 Date: Jan. 8, 2007

(30) Foreign Application Priority Data
Oct. 17, 2003 (DE) 103 48 344.6

(51) Int. Cl. A61L 2/04

(52) U.S. Cl. 422/1; 422/26; 422/292; 422/307

(57) ABSTRACT

The invention relates to a method and a device for the recooling of cleaned items in automatic cleaning and disinfecting machines for containers containing human excreta. According to the proposed method, the items to be cleaned, which are contained in a chamber (1), are rinsed off inside said chamber (1), after which a precleaning rinsing step takes place. The items to be cleaned, which are contained in the chamber (1), are then subjected to a final cleaning with water that contains a clear rinse additive, before the items to be cleaned are subjected to a disinfection step in the chamber (1) by introduction of steam into the latter. With the door (5) closed, air is forcibly introduced into the steam-filled chamber (1), which leads to steam condensation inside the chamber (1) and also to cooling and drying of the cleaned items contained in the chamber (1).

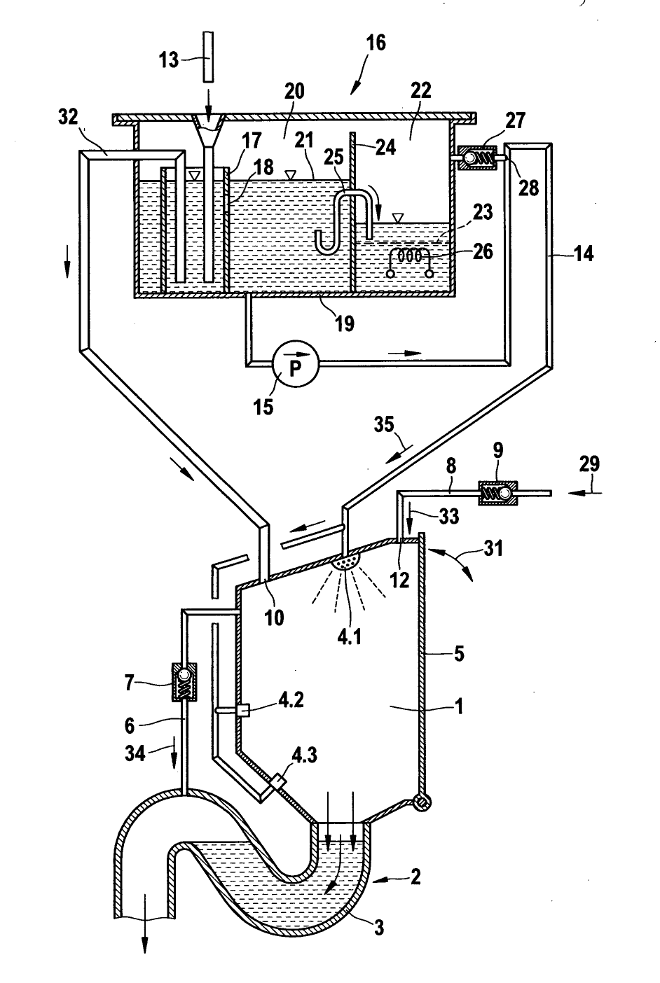

The invention relates to a method for the cooling of cleaned items in automatic cleaning and disinfecting machines, which are used for example in hospitals and nursing homes and the like. In addition to convenient handling and rapid operation, it is particularly important here to ensure thorough cleanliness and disinfection, i.e. hygiene.

### PRIOR ART

DE 195 09 877 C2 discloses a device for the cleaning and disinfecting of bedpans, urinals and the like. This device comprises a chamber, a steam-generating chamber, a water supply tank and a water pump, where a line for water intake into the chamber is designed as an overflow line of the water supply tank. The steam-generating chamber and the water supply tank are connected according to the principle of communicating vessels in such a way that, even with a low water level in the water supply tank, a steam generator in the steam-generating chamber is completely flooded, and a steam intake duct between steam-generating chamber and chamber likewise forms an overflow line of the water supply tank and the water supply tank forms an overpressure valve for the steam intake duct.

DE 198 31 950 C2 discloses a machine for the cleaning and disinfecting of medical ware. The medical ware is introduced through a door into a chamber, fixed in place there and sprayed with water or steam via nozzles. A water tank, a water inlet and a pump system for introducing cleaning fluid into a chamber are provided. A heater for the fluid is also used as a steam generator, the steam formed above the liquid level of the steam area being guided through a steam duct into the chamber. The water tank is divided by a partition arrangement into a main water tank and a steam area, down to below the lowest water level, while maintaining a water connection. The heater is arranged as steam generator in the steam area.

DE 198 38 180 C2 discloses a method and a device for the cleaning and disinfecting of containers. By means of this device, it is possible in particular to clean bedpans, urinals or suction bottles. The container in question is first emptied and undergoes a prerinse by continuous spraying with freshly supplied cleaning fluid and continuous removal of dirtied cleaning fluid via an outflow of the spray device. The spraying with cleaning fluid is interrupted, whereupon further removal of dirtied cleaning fluid takes place. Thereafter, the outflow of the rinsing device is closed. A predetermined quantity of cleaning or disinfecting fluid is introduced into the rinsing chamber. This is followed by continuous cleaning and disinfecting of the container by recirculation and spraying with the cleaning or disinfecting fluid contained in the rinsing chamber. Superheated steam is introduced into the rinsing chamber, and, during the introduction of the superheated steam, moist air is suctioned out of the rinsing chamber by means of a water jet pump or an electrically driven pump.

The disinfection step takes place with steam in the method known from DE 198 38 180 C2. By introducing steam into the rinsing chamber, the cleaned items are heated to a defined temperature. At the end of the disinfection step, the rinsing chamber is filled with steam and the cleaned items are heated to a temperature of for example 85° C. When the rinsing chamber is now opened by the operator, the steam escapes from the rinsing chamber into the work area. Depending on the disinfecting temperature, i.e. the temperature of the steam, the operator is at risk of being scalded or burned by the emerging steam; moreover, when the door of the rinsing chamber is opened, moisture is carried unnecessarily into the work area in which the automatic cleaning and disinfecting machine is located. Moreover, it is not readily possible for the operator to immediately remove the cleaned and disinfected items from the rinsing chamber without being exposed to the risk of burn injuries; a certain waiting time is generally required before the cleaned items that have been heated are cooled down by the cooler ambient air.

This situation has been remedied by water being injected into the rinsing chamber after the disinfection step, and the washed items being cooled in this way. Moreover, the injected water causes the steam in the rinsing chamber to condense. This additionally added water is taken from the built-in water supply tank of the automatic cleaning and disinfecting machine. A disadvantage of this is that the water from the supply tank may be charged with microorganisms and thus pose a risk of recontamination of washed material that has previously been disinfected. A further disadvantage of cooling by water introduced after the disinfection step lies in the increased water consumption. Moreover, the application of the water after the disinfection step makes the automatic drying of the heated items more difficult.

EP 1 032 432 B1 discloses a method of steam sterilization for treating sterile material, in which method, in a gas removal step using steam and a suction device, ambient air is first removed from a sterilization chamber. A steam sterilization step is then performed in which steam is introduced into the sterilization chamber and remains there at a predetermined temperature and at a certain pressure for a certain time. A non-condensable gas is then introduced into the chamber, as a result of which the remaining steam is displaced out of the chamber. In a subsequent step, the non-condensable gas is then pumped out of the chamber and an underpressure is generated in the chamber, with the material located in the chamber being dried. The pressure in the chamber is then returned to the ambient pressure and the material is removed from the chamber.

However, the method described in EP 1 032 432 B1 and the device for carrying out the described method have serious disadvantages. Thus, the method is reliant on a large number of valve-opening and pumping operations which follow one another in precise succession and which in practice necessitate the use of an elaborate pump system and complicated electronic valve control. The method and the device are therefore very time-consuming and expensive, such that the system cannot feasibly be used in many cases requiring cost-effective and rapid disinfection of hospital items, for example bedpans. Instead, a method and device would be desirable that manage without the use of expensive pump systems or electronically controlled shut-off valves.

The device and method described in EP 1 032 432 B1 have the further disadvantage that not enough consideration has been given to the need for a waste waster system, as exists particularly in the described hospital use. Since the described method is reliant on complex vacuum stages, the waste water system has to be separated from the actual sterilization chamber by a boiler bottom valve, since otherwise fluid would get into the pump system. The system is therefore only suitable for small amounts of waste water, and not, for example, for direct cleaning of bedpans. The device and the method described in EP 1 032 432 B1 are therefore mainly suitable for the sterilization of smaller medical items such as operating instruments, for example.

For daily hospital requirements, by contrast, it would be desirable to make available a method and a system that can also cope with large amounts of liquid without difficulty, in other words that have a more efficient waste water system. However, an aim in this connection must be to prevent the odors which are caused by the large amounts of waste water and which are unavoidable in the device described in EP 1 032 432 B1, where air from the sterilization chamber is let out directly into the environment.

### DISCLOSURE OF THE INVENTION

In view of the disadvantages of the solutions known from the prior art, the object of the invention is to avoid the danger of recontamination of items that have been cleaned and disinfected, to reduce the water consumption of the automatic cleaning and disinfecting machine per work cycle, and to improve the working conditions for the person operating the automatic cleaning and disinfecting machine.

According to the invention, this object is achieved by the features of patent claim 1.

According to the proposed solution, the program steps, namely rinsing with cold water, precleaning with hot water (or cold water), cleaning with clear rinse solution, and heat disinfection with steam, proceed in a known manner inside the automatic cleaning and disinfecting machine. According to the invention, after the disinfection of the cleaned items by means of steam introduced into the chamber, air from the environment, for example from the work area in which the automatic cleaning and disinfecting machine is located, is blown forcibly into the chamber. In relation to the conditions in the chamber, the air drawn in from the work area is cold and dry. When this air enters the steam-filled chamber of the automatic cleaning and disinfecting machine, the steam condenses in the latter, and the cleaned items contained in the chamber cool. The air delivered from outside, for example ambient air, is led off into the waste water system, together with the remaining steam, through an outflow pipe which can be connected to the waste water system of the automatic cleaning and disinfecting machine.

To influence the flow of air inside the chamber of the automatic cleaning and disinfecting machine, shut-off elements such as slides, flaps and valves can be fitted in the lines leading away from and to the chamber. Chamber hereinafter designates the chamber of an automatic cleaning and disinfecting machine in which the items to be cleaned are treated. The shut-off elements in the lines leading away from and to the chamber can be designed, for example, as a nonretum valve, which can be fitted between the chamber and the fan carrying in the air. Moreover, the shut-off elements, designed for example as flaps, slides or valves, can be used to avoid odors emanating from the waste water system during the periods when the automatic cleaning and disinfecting machine is shut down.

If the stream of air introduced into the chamber is maintained in the latter after the condensation of the steam, an additional effect that can be achieved is that the stream of air introduced takes up moisture from the surface of the cleaned items and of the chamber and dries them despite the chamber door being closed.

By means of the method proposed according to the invention, it is possible to avoid recontamination of the washed items. The ambient air blown into the chamber, i.e. with ambient air moisture and at ambient air temperature, is sterile compared to the water present in the water tank, so that the danger of recontamination by microorganisms contained in the water is ruled out. The water additionally injected into the chamber in accordance with the previous solution and the associated increase in water consumption are avoided, since another medium, namely ambient air, is used for condensing the steam in the chamber. Moreover, the solution proposed according to the invention largely excludes the possibility of plumes of vapor escaping into the work area after the chamber door is opened and impairing the work conditions of the person operating the automatic cleaning and disinfecting machine.

Moreover, the solution proposed according to the invention advantageously ensures that the cleaned items are dried inside the chamber with the door closed and, therefore, no additional moisture can get into the work area where the automatic cleaning and disinfecting machine is located. The fact that air is forcibly blown into the chamber after completion of the heat disinfection step ensures, on the one hand, that the steam condenses, and, on the other hand, that the cleaned and disinfected items contained in the closed chamber are cooled. In this connection, the solution proposed according to the invention can ensure that the cleaned and disinfected items are cooled to a temperature which is such that they can be removed from the chamber by the operating person, without the latter suffering bums or scalds when taking hold of the cleaned items.

### DRAWING

The invention is described in more detail below with reference to the drawing:

The single FIGURE is a schematic representation showing the structure and the duct system of an automatic cleaning and disinfecting machine that can be operated using the method proposed according to the invention.

In an automatic cleaning and disinfecting machine used in a hospital or nursing home, bedpans, urinals and other collecting containers for human excreta are cleaned and disinfected in a chamber 1. These containers can be disinfected by means of heat, i.e. with steam, or also by chemical means.

For this purpose, a chamber 1 is generally formed in the automatic cleaning and disinfecting machine, which chamber 1 can be loaded from the outside, via a door 5, with the containers that are to be cleaned, and through which the cleaned and disinfected containers can also once again be removed from the chamber 1.

At its lower end, the chamber 1 comprises an outflow 2 which contains a siphon bend 3 and via which the remains of human excreta can be conveyed into a waste water system. The outflow 2 at the lower end of the chamber 1 opens into an outflow system. To avoid formation of odors in the chamber 1, the siphon bend 3 formed in the outflow 2, and generally provided as an odor barrier in waste water systems, also serves the same purpose in the automatic cleaning and disinfecting machine described here. The chamber 1 of the automatic cleaning and disinfecting machine is accessible via a pivoting door 5. The door 5 can be moved via a hinge fitted at the lower end of the door and is able to move in the opening/closing direction 31 in accordance with the double arrow shown in the drawing. The chamber 1 can be acted upon by steam via a water/steam unit 16. The drawing shows nozzles 4.1, 4.2, 4.3 from which steam can enter the chamber 1. These nozzles are integrated into the roof surface of the chamber 1, but can also be provided in its side surfaces. The nozzles 4.1, 4.2, 4.3 through which steam can be conveyed into the chamber 1 could equally well be mounted on the back wall of the chamber 1. Also opening into the chamber 1, at a mouth 10, there is a safety overflow 32 via which water from the water/steam unit 16 in the upper area of the automatic cleaning and disinfecting machine can flow over into the chamber 1 and pass from there into the outflow 2. The safety overflow 32 could also open into the outflow 2. The mouth 10 of an intake air duct 8 is also situated in the roof surface of the chamber 1 of the automatic cleaning and disinfecting machine. Finally, air is led from the chamber 1 into the outflow 2 via an exhaust air duct 6, the mouth of the exhaust air duct 6 lying behind the siphon bend 3 of the outflow 2.

A water/steam unit 16 is received in the upper area of the automatic cleaning and disinfecting machine according to the drawing. The water/steam unit 16 is assigned a water pump 15 which is arranged below the bottom 19 of the water/steam unit 16. By means of the water pump 15, which increases the water pressure, water is pumped out of a water tank 20 of the water/steam unit 16 through a water supply line 14 and into the chamber 1. The water tank 20 of the water/steam unit 16 is supplied via a cold water or hot water intake 13. Depending on how the device is connected up to the building, either cold water in a temperature range of between 10° C. and 30° C. can be introduced into the water tank 20 via the water intake 13 or, if it is connected to a hot water source, water at a temperature of between 45° C. and 60° C. can flow into the water tank 20 of the water/steam unit 16. It is also possible for the water tank 20 to be filled with a mixture of hot and cold water. The hot water or cold water entering via the water intake 13 flows into a pot 17 secured on the bottom 19 of the water/steam unit 16. The water level in the pot 17 lies below an overflow 18 in the state shown in the drawing. The water flowing into the pot 17 via the water intake 13 flows constantly through the overflow 18 into the water tank 20. In the case of excess admission of water, water from the pot 17 flows through the safety overflow 32 and through the mouth 10 into the chamber 1 and then flows through the always open outflow 2 into the waste water system, so that no water damage can occur in the room in which the automatic cleaning and disinfecting machine is located.

The water flowing from the pot 17 into the water tank 20 via the overflow 18 fills the water tank 20 up to a water level 21. The water tank 20 is separated by a partition wall 24 from a steam generator 22 of the water/steam unit 16. For filling the steam generator 20 with water, an overflow line 25 extends through the partition wall 24. Water flows into the steam generator 22 via the overflow line 25. After water has been pumped off, a level 23 is established in the steam generator 22. To heat the water present in the steam generator 22, the supply of water contained in the steam generator 22 is heated by a heater 26, symbolized by a coil in the drawing. The steam that arises as the water is heated is conveyed through a duct section into the supply line 14 that leads from the water pump 15 to the chamber 1. The duct section between the steam generator 22 and the supply line 14 to the chamber 1 is closed by a nonreturn valve 27. Because of the pressure of the steam in the steam generator 22, the nonreturn valve 27 is able to open, so that steam at an outlet mouth 28 can flow into the supply line 14 to the chamber 1.

Water conveyed from the water tank 20 via the pump 15 can also be injected into the chamber 1 via spray nozzles 4.2, 4.3 arranged in the back region of the chamber 1. The spray nozzles 4.1, 4.2, 4.3 can also be arranged on the side walls or on the face of the door that closes the chamber 1, that is to say on the face directed towards the chamber 1.

With suitable generation of steam in the steam generator 22, the steam needed for the heat disinfection of the cleaned items contained in the chamber 1 is introduced through the opened nonreturn valve 27 and the outlet mouth 28 into the line 14 and is introduced into the chamber 1 via nozzles 4.1, 4.2, 4.3 integrated in the roof surface of the chamber 1 or via nozzles 4.1, 4.2, 4.3 integrated on the back wall of the chamber 1.

Ventilation of the chamber 1 is also possible via an intake air duct 8 which can be opened and closed via a intake air valve 9. Intake air 29, which is relatively sterile ambient air at ambient air conditions, is introduced into the chamber 1 via a fan (not shown in the drawing). The direction of admission of the ambient air 29 is indicated by reference number 33.

The program running in the chamber 1 of the automatic cleaning and disinfecting machine can be executed in accordance with the method steps outlined below; it is not essential to run right through the method steps indicated below, and it is quite possible for the method steps represented below also to be supplemented by other method steps:

First, the chamber 1 is loaded with containers, for example bedpans, urinals, chamber pots or the like, which are soiled with human blood or with other human excreta, in order to clean these containers and use them again. After the chamber 1 has been loaded, the door 5 is closed and a first rinsing step with cold water is carried out. By way of the spray nozzles 4.2, 4.3, the containers held in the inside of the chamber 1 are rinsed with cold water in a temperature range of between 10° C. and 30° C. Depending on the cleaning program running in the chamber 1, said rinsing step with cold water can be followed by a second cleaning step that can be carried out with hot water, this water having a temperature of between 45° C. and 60° C. The second precleaning step can by contrast also be carried out with cold water in the aforementioned temperature range, in order to ensure that protein residues that may be contained in the human excreta do not adhere to the items that are to be cleaned and disinfected.

The second precleaning step carried out on the containers held in the chamber 1 can be followed by a final cleaning with a clear rinse. For this purpose, a clear rinse additive can be admixed to the wash water via a fine-dosing pump (not shown in the drawing). The clear rinse additive is added in small amounts to the water conveyed from the water tank 20 via the water pump 15, whether cold water or hot water, and applied to the containers that have been cleaned by the first rinsing step and the second precleaning step. The cleaning step using addition of a clear rinse agent is followed by a heat disinfection step. In the heat disinfection step, the removal of cold or hot water from the water tank 20 of the water/steam unit 16 is interrupted, and steam that has been generated in the steam generator 22 is introduced via the nonretum valve 27 into the supply line 14 to the chamber 1. The steam emerges either at nozzles 4.1, 4.2, 4.3 integrated in the roof surface 4 of the chamber 1 or at nozzles 4.1, 4.2, 4.3 which are integrated in the back wall of the chamber 1. The steam permits disinfection of the containers in the chamber 1 that have previously been cleaned by the cleaning steps outlined above. During all of the steps listed above, the door 5 of the chamber 1 is at all times in the closed state. After the disinfection step, the chamber 1 is completely filled with steam because the door 5 is closed, only some of which steam is able to escape through the exhaust air duct 6. After the disinfection step, the intake air valve 9 in the intake air duct 8 is opened and ambient air 29 at ambient air conditions is introduced into the chamber 1. Compared to the steam present in the chamber 1, the ambient air 29 is cold and dry. As a result of this, the steam contained in the chamber 1 condenses, and the cleaned items contained in the chamber 1 cool down. Together with the residual steam contained in the chamber 1, the delivered ambient air 29 is conveyed through the exhaust air duct 6, in which the exhaust air valve 7 is opened, into the outflow 2 and thus removed from the chamber 1. To influence the flow of intake air into the chamber 1, additional elements such as slides, flaps, valves 9 or the like can be fitted in the intake air duct 8 and in the exhaust air duct 6. If the flow of ambient air 29 in the direction of admission 33 into the chamber 1 is maintained, a further added effect obtained is that the stream of intake air forcibly blown into the chamber 1 picks up moisture from the surface of the items to be cleaned and of the wash chamber and, despite the door 5 being closed, dries the surface of said items and of the wash chamber. By this means, it is possible to ensure that, after completion of the wash cycle and when the door 5 shutting off the chamber 1 is opened, plumes of steam do not escape which, under unfavorable circumstances, can lead to scalding or burning of the hands and arms of the operator. It has been found that, for the fan received in the intake air duct 8 for drawing ambient air 29 into the chamber 1, an operating time of approximately 40 seconds is sufficient to allow condensation of the steam present in the chamber 1 after the heat disinfection. A prolongation of the operating time of the fan in the intake air duct 8 can be utilized to additionally provide for drying of the cleaned items contained in the chamber 1 and not only to cool these to a temperature allowing them to be handled, but to cool them to temperatures below this, for example below 65° C. For the fan conveying ambient air 29 or air from another air source into the chamber 1, the operating time can be adjusted according to requirements, but the operating time should be at least long enough to guarantee that the steam contained in the chamber 1 has completely condensed.

Although not shown in detail in the drawing, the admixture of the clear rinse additive takes place via a separate pump which, in addition to the water pump 15, can be assigned to the water/steam unit 16, and the line acted upon by this additional dosing pump opens into the line 14 to the chamber 1.

Moreover, in a departure from the cleaning program outlined above, and in order to remove firmly adhering residues such as ointment residues, for example, which may remain on the seat surfaces of containers receiving human excreta, it is entirely possible to add to the cleaning liquid an additive that removes ointments. Besides an additional program step, for example the addition of an ointment-removing agent to the cleaning liquid, additional further steps can also be incorporated into the cleaning program, just as it is also possible for the cleaning steps outlined above to be freely combined according to requirements either with cold water or with hot water in the stated temperature ranges.

The measure proposed according to the invention, and involving introduction of ambient air 29 under ambient air conditions into the chamber 1 after completion of the heat disinfection with steam, ensures a considerably reduced risk of the previously cleaned and disinfected items in the chamber 1 being recontaminated by unclean water possibly present in the water tank 20. Moreover, the measures proposed according to the invention afford the possibility of cooling the cleaned and disinfected items contained in the chamber 1 such that they can easily be removed from the chamber 1 by the operator, and, with suitable prolongation of the operating time of the fan held in the intake air duct 8, it is possible to achieve almost complete drying of the cleaned items contained in the chamber 1.

Since the water container 20 is filled either with cold water or with hot water via the inlet from the building, it is not possible to entirely rule out the possibility of microbial contamination of this water. However, this water is still suitable for carrying out the first step of rinsing and the second step of precleaning, but not for recooling the cleaned items after they have been heat-disinfected with steam. Moreover, the introduction of ambient air 29 into the chamber 1 after completion of the disinfection step, as proposed according to the invention, affords the advantage of making it possible to save on the solutions known from the prior art for recooling the water that has been used. Moreover, the solution proposed according to the invention means that, because the door 5 of the chamber 1 can be kept closed, it is possible to avoid the escape of plumes of vapor into the work area in which the automatic cleaning and disinfecting machine is located. Moreover, the introduction of ambient air 29 after completion of the disinfection step with steam affords the possibility of drying the cleaned items contained in the chamber 1 with the door 5 closed.

1. A method for the cooling of cleaned and disinfected items contained in a chamber (1) of an automatic cleaning and disinfecting machine that has an outflow (2), the cleaned items being disinfected by heat, and a washing or cleaning program with a variable sequence of program steps being executed inside the automatic cleaning and disinfecting machine, said method comprising the following method steps:
a) the final cleaning of the items contained in the chamber (1) is carried out using water with addition of auxiliary agents,
b) the cleaned items contained in the chamber (1) are disinfected by heat,
c) after the heat disinfection of the cleaned items, air (29) is forcibly introduced into the closed chamber (1), and
d) with the door (5) of the chamber (1) closed, exhaust air is conveyed out of the closed chamber (1) into the outflow (2).

2. The method as claimed in claim 1, characterized in that, in method step d), with the door (5) of the chamber (1) closed, moist exhaust air is conveyed out of the chamber (1) via an exhaust air duct (6).

3. The method as claimed in claim 1, characterized in that, in the event of a prolonged duration of the removal of moist exhaust air out of the chamber (1) with the door (5) closed, an additional drying of the cleaned items contained in the chamber (1) takes place.

4. The method as claimed in claim 1, characterized in that the air introduced into the chamber (1) after the disinfection step is ambient air.

5. The method as claimed in claim 1, characterized in that automatic shut-off elements (7, 9) are provided in the exhaust air duct (6) and in the intake air duct (8), respectively, for controlling the flow of intake air and exhaust air.

6. The method as claimed in one or more of patent claims 1 through 5, characterized in that the air admitted through the air intake duct (8) of the chamber (1) is guided through a microfilter in order to improve the sterility.

7. A device for carrying out the method as claimed in one or more of claims 1 through 6, characterized in that a chamber (1) can be acted upon both by cold water or hot water and also by steam from a water/steam unit (16), the cold water/hot water being introduced via spray nozzles (4.1, 4.2, 4.3), the chamber (1) being connected to an outflow (2) via an exhaust air duct (6), and the chamber (1) having an outlet mouth (12) of an openable or closable intake air duct (8) via which the chamber (1) in the closed state can be acted upon by air.

8. The device as claimed in claim 7, characterized in that the outlet mouth of the supply line (14) via which the chamber (1) can be acted upon by steam simultaneously via nozzles (4.1, 4.2, 4.3) is formed either on a roof surface of the chamber (1) or on a back wall or in the lower area or the side walls of the chamber (1).

9. The device as claimed in claim 7, characterized in that automatic shut-off elements which are either spring-controlled or weight-controlled or membrane-controlled or controlled by differential pressure or designed as nonreturn valves (7, 9) are received in the lines (6, 7) for intake air or exhaust air connected to the chamber (1).

10. The device as claimed in claim 7, characterized in that the shut-off elements are designed as forcibly controlled shut-off elements.
